(19) United States
(12) Patent Application Publication
(10) Pub. No.: US 2003/0163149 A1
(43) Pub. Date: Aug. 28, 2003

(54) Breath freshener lollipop

(76) Inventor: Charles Gilbert Heisinger JR., Evanston, IL (US)

Correspondence Address: Charles G. Heisinger, Jr., 1328 Washington Street, Evanston, IL, 60202, US

(21) Appl. No.: 10/197,365

(22) Filed: Jul. 17, 2002

Related U.S. Application Data
(60) Provisional application No. 60/359,776, filed on Feb. 26, 2002.

(51) Int. Cl. A61B 17/24

(52) U.S. Cl. 606/161

(57) ABSTRACT

A lollipop for breath freshening and of the type having an applicator head (12) affixed to a handle (14). The applicator head (12) is made of an edible substance (21) intermixed with an antibacterial means (19) and molded with a textured surface (24) for separating the folds of the tongue and accessing the grooves of the tongue for the purpose of eliminating the precursors of Volatile Sulfur Compounds.

### CROSS REFERENCES TO RELATED APPLICATIONS

U.S. Pat. No. 5,735,864 Disposable Tongue Cleaner Provisional Patent App. No. 60/359,776 Disposable Antibacterial Tongue Applicator, filed Feb. 26, 2002.

### BACKGROUND OF THE INVENTION

This invention relates to breath fresheners, specifically to such fresheners in the form of lollipops which are used to eliminate the precursors of Volatile Sulfur Compounds.

The existence of bad breath has long been a serious problem. The reasons for its occurrence have not been fully understood, and there have been many ill-conceived attempts to overcome its effects. Bad breath is caused primarily by volatile sulfur compounds (VSCs). VSCs are formed by anaerobic bacteria that metabolize protein. Research indicates that VSC formation occurs primarily on the tongue dorsum.

The human tongue has folds and grooves in its dorsal surface and the VSCs that are encountered reach the posterior surface area of the tongue, as well as other areas in the oral cavity, and find their way particularly into those folds and grooves and become firmly lodged therein. It is very difficult to dislodge these materials. Heretofore, most efforts, if not all, to physically remove those materials, involved devices that were mechanical in nature, and used to mechanically remove the materials. However, purely mechanical devices are not very effective for the purpose.

Numerous inventors have designed tongue cleaners, as evidenced by the following prior patents.

U.S. Pat. No. 1,891,864 Barnett

U.S. Pat. No. 2,218,072 Runnels

U.S. Pat. No. 5,957,942 Yudelman

U.S. Pat. No. 5,967,152 Rimkus

U.S. Pat. No. 6,015,293 Rimkus

U.S. Pat. No. 6,032,315 Liebel

U.S. Pat. No. 6,056,763 Parsons

U.S. Pat. No. 6,139,558 Wagner

U.S. Pat. No. 6,352,545 Wagner

U.S. Pat. No. 6,402,768 Liebel

The primary objective of the cleaners in those prior patents is to effect a mechanical removal of material and debris from the dorsal surface of the tongue. As such, a prominent feature of the device is a scraper, which serves to relocate debris from the surface of the tongue. To the extent that a tongue scraper merely wipes debris off of the surface of the tongue and that precursors of VSCs remain lodged in the folds and grooves of the tongue, scrapers themselves, alone, fail to attack a major cause of bad breath.

Some tongue cleaners have employed brushes and sponges as elements of their design. For example:

U.S. Pat. No. 5,226,197 Nack et al.

U.S. Pat. No. 6,083,235 Wagner

Brushes lack the surface area to serve as a delivery or removal means for a substantial amount of antibacterial material. Sponges, impregnated with antibacterial agent, tend to absorb the agent rather than dispense it.

There are several widely recognized compounds that markedly inhibit VSC formation that are made from zinc, copper or chlorine. Chlorine dioxide, a chlorine-based compound, is unstable and is limited to high-end dentist prescribed, mouth rinses. Divalent Zinc or copper ions, when chelated or as Acetate or as Gluconate, can be used in products such as hard candy, or lozenges or tablets. Most of the consumer mass-marketed breath-freshener products (gums, mints, rinses, pastes, strips) do not contain divalent elements. They don't eliminate the source of bad breath, they just mask breath problems.

As will be seen in the following additional description of the entire device, and the various aspects thereof, an important object of the present invention is to eliminate precursors of VSCs residing in the folds and grooves of the tongue. This is done by producing a chemical action that counteracts the formation of the VSCs. While there is a mechanical action in the present case, that action is not relied on solely for eliminating the precursors, but serves as one step in cooperation with the chemical action that is produced in eliminating the precursors of the VSCs.

In previous products where zinc or similar divalent, VSC inhibiting compounds are used, delivery to the posterior surface of the tongue is often lacking. Gag reflex prevents many products, such as lozenges or standard-size tablets for accessing the back of the tongue. Even if an antibacterial agent is successfully applied to the back of the tongue, it may remain on the surface, where it is unable to penetrate inner folds and grooves of the tongue.

There are several products on the market that include a cleaner and a separately packaged antibacterial preparation. This application is both inconvenient and messy.

Background of the Invention—Objects and Advantages

Other detailed objects and advantages of the invention are:

(a) to provide an applicator which is portable and convenient to use;

(b) to provide an applicator which is disposable;

(c) to provide an applicator which is sanitary due to its single-use design;

(d) to provide an applicator which is easy and inexpensive to manufacture;

(e) to provide an applicator which does not cause gag reflex when moved to the posterior of the tongue;

(f) to provide an applicator which is easily packaged for sale;

(g) to provide an applicator which incorporates an antibacterial means as its applicator head;

(h) to provide an applicator which efficaciously dispenses an antibacterial means;

(i) to provide an applicator that brings an antibacterial means in prolonged contact with volatile sulfur compound precursors residing in the folds and grooves of the dorsal surface of the tongue, through deliberate, controlled, and precise positioning of the device.

An additional and important summary object is to eliminate precursors of VSCs residing in the folds and grooves of the tongue. This is done by producing a chemical action that counteracts the effects of the VSCs. While there is a mechanical action in the present case, this action serves to facilitate the chemical action that is produced in eliminating the precursors of the VSCs.

This means for chemically counteracting the precursors, includes zinc or copper, as described in detail hereinbelow.

### BRIEF DESCRIPTIONS OF THE INDIVIDUAL FIGURES OF THE DRAWINGS

FIG. 1 is a perspective view of a device made according to the present invention.

FIG. 2 is a face view of a first side of the device.

FIG. 3 is a face view of a second side thereof, opposite that side shown in FIG. 2.

FIG. 4 is a sectional view taken at line 4-4 of FIG. 2.

FIG. 5 is a diagrammatic view representing the step of molding the device.

FIG. 6 is a view oriented according to FIG. 2 showing a modified form of device.

FIG. 7 is a face view, similar to FIG. 2 of a modified form of the device.

FIG. 8 is a sectional view, similar to FIG. 4 showing another modified form of the device.

### DETAILED DESCRIPTION

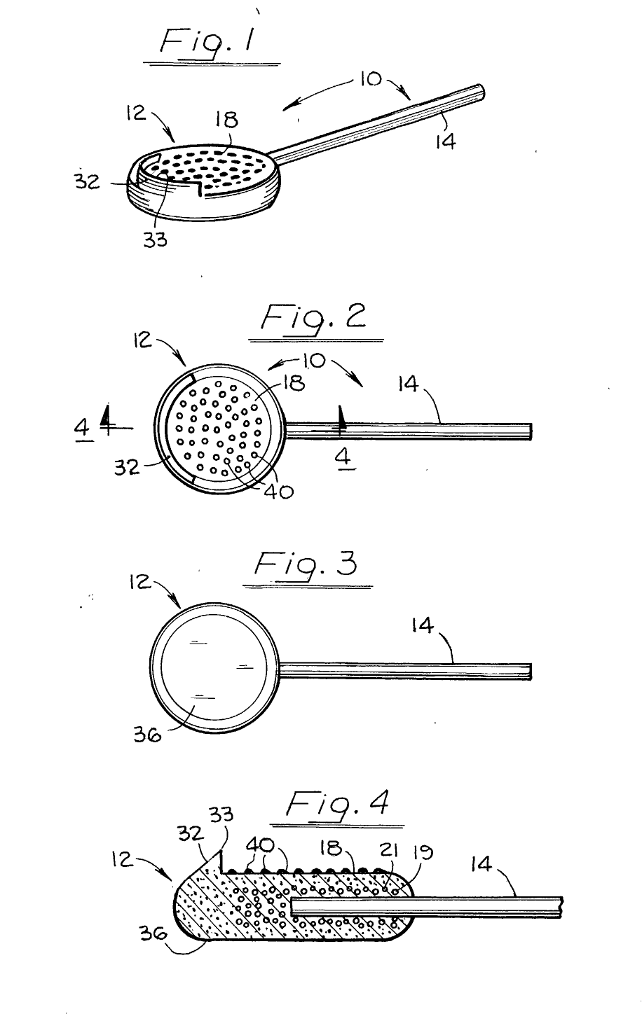

A device 10 embodying the invention is shown in FIGS. 1-4 and includes an applicator head 12 and a handle 14. The head 12 has a first, of front, surface 18, displayed in FIGS. 1 and 2 which is rough in texture. It is also provided with an integrated scraper 32 (FIGS. 2 and 4) on the front side. The scraper is a continuous rib element having a sharp edge 33 (FIG. 4), and of a predetermined depth, approximately 1/16 to ⅛ inch, extending about one-third the periphery of the head at a position opposite the inner end of the handle. The head has a second or back surface 36 that is preferably flat and smooth.

The texture 40 of the front surface 18 (FIGS. 2 and 4) is represented diagrammatically by the elevated points on the surface. This surface may be for example similar to a texture found on sand paper, which may be any of various degrees of coarseness, however not as abrasive as sandpaper.

The device is produced in a manner similar to ordinary lollipops. In the formation of the device an antibacterial means 19 consisting of a divalent element, ie. zinc or copper, as Acetate, Gluconate, or in chelated form, is intermixed with an edible substance 21 and distributed throughout the mass thereof. This material is difficult to illustrate in the drawings, but reference is made to FIG. 4, which shows relatively large, and small, dots representing the, said antibacterial means and said edible substance, respectively, which are shown distributed throughout the applicator head.

FIG. 5 depicts the molding process of the device 10. The edible substance 21 and antibacterial means 19 (FIG. 4) are poured into a mold 16 and permitted to harden inside the mold, thereby assuming the shape of the mold. The first, or front, surface 18 of the head, displayed in FIGS. 1 and 2 is rough in texture, and is formed by a corresponding surface 28 in a mold 16 depicted of FIG. 5. The integrated scraper 32 and sharp edge 33 introduced in FIG. 1 is formed by a cavity 34 in the mold. Mold surface 30 forms the back surface 36 in FIG. 3. The handle 14 is molded therein, in integrated form. The handle is in the form of a stick, or suitable material such as plastic or wood, or of tightly wound rolled paper.

In the use of the device, the user inserts the applicator head into the mouth, and applies the first side 18 against the tongue. That surface engages the tongue substantially throughout the area of the surface and the device is moved on the tongue moving the applicator head in a brushing or rubbing action thereon. This movement may be from back to front, or laterally, or in most cases both directions, and the roughened or abrasive surface 18 scrapes the surface of the tongue, loosening the material that accumulated on the tongue.

In this same action, the scraper 32 also scrapes over the surface of the tongue and it more often is positioned near the rear of the tongue, or the dorsum. The scraper produces a more abrasive or scraping action on the tongue, than does the surface 18, and specifically loosens the folds and exposes the grooves in the tongue.

As the device is held in the mouth, the material of the applicator head is dissolved, principally by the action of the saliva, and this forms a definite liquid in the oral cavity. As the material of the applicator head so dissolves, the zinc or copper material is released and neutralizes the VSC precursors. The resulting material (liquid) is chemically neutral, and can be swallowed.

The particular details of the mechanical construction of the device may vary greatly. For example in the device of FIGS. 1-4, the scraper 32 extends less than half of the circumference of the head, enabling the roughened surface 18 to engage the front portion of the tongue.

As shown in FIGS. 1-3, the scraper is near the far or inner end of the device so that when it is in the usual position of consumption, the scraper is at the inner end of the tongue and engages it, and when the device is moved the scraper easily wipes debris on the tongue.

However other shapes and sizes of applicator head may be utilized. For example in FIG. 6 the device 37 has an applicator head 38 which may be round and flat as in FIGS. 1-4. It may be desired also to provide more scraping effect, as opposed to an abrasive effect by a roughened surface, and accordingly, a plurality of scrapers or scraper elements 48 are provided, which may be each of shorter lengths, and positioned on a front surface 42 of the applicator head in a distributed arrangement, covering the greater portion of the area of the front side. This device also assures engagement with the far inner end of the tongue.

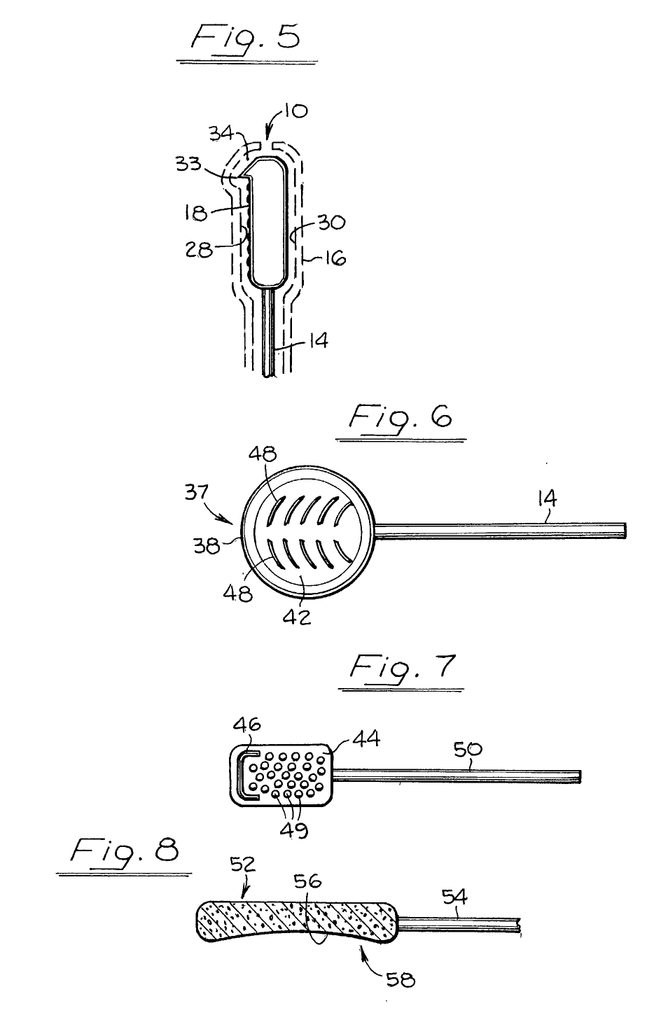

FIG. 7 shows another form of applicator head 44 which is rectangular in shape, and shorter than the diameter of the previous two forms. In this case the scraper 46 may be positioned at the far inner end of the applicator head, and a roughened surface or abrasive surface 49 may be formed on various portions of the remainder of the applicator head. The handle 50 is included in this form also.

FIG. 8 shows another form of the applicator head 52 in which a handle 54 is secured. This applicator head may be of any desired outline shape such as round, polygonal, etc but it is provided with a concave surface 56 on its first side 58. This concave surface provides an advantage in engaging the dorsal surface of the tongue. The dorsal surface of the tongue is usually more curved, presenting a convex surface, and the abrasive surface 56 thereby more easily engages the convex surface of the tongue.

### SUMMARY

In the case of all of the different forms of the device, the handle is a great advantage in controlling the applicator head. The handle enables the user to move the applicator head across the tongue more easily, and assures that it engages portions of the tongue that may not be engaged by a head without a handle. Additionally, the handle may position the applicator head over a stationary location on the tongue for a prolonged period of time, thereby permitting the antibacterial means to become dissolved on the hard-to-reach posterior surface of the tongue.

Another great advantage of providing the handle attached to the applicator head, is that the applicator head will not readily be plunged into the throat as a result of a reflex gag phenomenon.

Upon depletion of the applicator head, due to dissolution thereof, the handle can be discarded.

As the applicator head is dissolved, the material thereof forms a fluid, or liquid condition, and is swallowed.

In the dissolution of the candy material, the ingredients of zinc or copper are released from the body of the applicator head and come in contact with the VSCs, neutralizing the latter, and the resulting fluid can be, and is expected to be, swallowed.

The entire device is extremely simple and inexpensive, and is also attractive due to the popular taste of the candy material.

Additional Embodiments:

In addition to the antibacterial means, the applicator head 12 may comprise suitable natural or artificial flavorants or colorants, such as peppermint oil, menthol, spearmint oil, vanilla, cinnamon, wintergreen oil, fruit flavorings including but not limited to lemon oil, orange oil, grape flavor, grapefruit oil, apple, apricot essence and combinations thereof.

Suitable food colorants include beta-carotene, turmeric, and other USFDA-approved dyes suitable for food applications.

Saliva from the tongue comes in contact with the textured surface, causing the antibacterial medicament to dissolve and further contact the folds and grooves of the tongue. Divalent elements in the medicament interact with the precursors of the VSCs, retarding their formation. The cleaner is occasionally pulled forward toward the anterior portion of the tongue, thereby clearing debris from the posterior portion of the tongue. When the textured surface has been fully dissolved, the balance of the lollipop may be sucked on, circulating flavorants and fresheners from the applicator head throughout the oral cavity.

Ease of Manufacture: The lollipop applicator is manufactured using standard techniques known in the candy and confectionery industry. A special mold is created to form the textured surface and scraper.

Single-use Function: The single-use function alleviates the need for between-use storage, and presents the user with a clean, sanitary device for each use. The dissolution of the applicator head during use leaves only a handle or stick to discard.

Although the description above contains many specifications, these should not be construed as limiting the scope of the invention, but merely providing illustrations of some of the presently preferred embodiments of the invention. In particular, the placement on the applicator head of one or more textured surfaces and zero or more scrapers may have numerous variations.

Thus the scope of the invention should be determined by the appended claims and their legal equivalents, and not only by the examples given.

Another form of the mechanical construction of the device may include an applicator head that is formed by pressing material under extremely high pressures. This dense head will dissolve slowly, and correspondingly the abrasive surface will also dissolve slowly, retaining the ingredients therein (zinc, copper) to good effect over a longer period of time.

I claim:

1. An oral-hygiene device, in the form of a lollipop, for applying antibacterial medicament to eliminate volatile sulfur compound precursors on the tongue comprising,
an elongated handle having a proximate end for grasping in either hand,
and a distal end,
an applicator head attached to said distal end of the handle,
said applicator head being comprised of an edible substance,
wherein, said applicator head is provided with a textured surface for opening folds and accessing grooves on the dorsal posterior surface of the tongue, and includes antibacterial medicament means for eliminating volatile sulfur compound precursors existing therein,
whereby, said device, in response to repeated movements thereof over the tongue, temporarily separates folds and accesses the grooves on the tongue, thereby permitting said antibacterial medicament to come into contact with precursors of volatile sulfur compounds and thereby inhibiting volatile sulfur compound formation.

2. The device of claim 1 wherein, said handle is comprised of any of,
a) wood,
b) thermoplastic,
c) tightly would paper.

3. The device of claim 1 wherein,
the applicator head is a pressed tablet.

4. The device of claim 1 wherein,
the applicator head is principally a hard candy.

5. The device of claim 1 wherein,
the applicator head includes any of the following flavorants,
a) peppermint,
b) menthol,
c) spearmint,
d) vanilla
e) cinnamon,
f) wintergreen oil,
g) Lemon oil,
h) orange oil,
i) grape oil.

6. The device of claim 1 wherein,
said antibacterial means includes any of the following divalent elements,
a) zinc,
b) copper,
c) chlorine.

7. The device of claim 1 wherein,
the applicator head includes any of the following colorants,
a) beta carotene,
b) turmeric,
c) and FDA-approved coloring agent.

8. The device of claim 1 wherein,
the applicator head includes a scraper at a different location from that of the textured surface.

9. A device for cleaning the tongue and freshening the breath, comprising,
an elongated main member having a perimeter edge and having a substantially flat textured surface for accessing the folds and grooves of the dorsal surface of the tongue, and
including an antibacterial medicament which is formed into a textured surface,
a handle attached to said member for grasping by either hand,
whereby said device, in response to movement thereof over the tongue, temporarily separates the folds and pockets and accesses the grooves on the tongue, and allows the antibacterial medicament to come into contact with precursors of volatile sulfur compounds that are lodged in said folds and grooves of the tongue.

10. The device of claim 9, wherein,
said textured surface is sandpaper-like in construction.

11. The device of claim 9, wherein,
the applicator head includes a scraper extending along said perimeter of the applicator head at the distal end of the applicator head.

12. The device of claim 1, wherein,
the applicator head includes a generally flat surface, and includes a plurality of scrapers on said surface.

13. A method for neutralizing volatile sulfur compound precursors, comprising,
accessing said precursors on the posterior dorsal surface of the tongue through the deliberate, controlled, and precise positioning and scrubbing action of a substantially flat textured surface on an applicator head comprised of an edible substance and an antibacterial medicament, including a divalent element which retards the formation of precursors of volatile sulfur compounds.
